(19) United States
(12) Patent Application Publication
(10) Pub. No.: US 2018/0050164 A1
(43) Pub. Date: Feb. 22, 2018

(54) Portable Vaporizer

(71) Applicant: Resolve Digital Health Inc., Concord (CA)

(72) Inventor: Rob ADELSON, Vaughan (CA)

(73) Assignee: Resolve Digital Health Inc., Concord (CA)

(21) Appl. No.: 15/671,643

(22) Filed: Aug. 8, 2017

Related U.S. Application Data
(60) Provisional application No. 62/375,591, filed on Aug. 16, 2016.

(51) Int. Cl. A61M 15/00

(52) U.S. Cl. CPC A61M 15/008; A61M 15/0021; A61M 15/0086; A61M 2205/587; A61M 2205/8206; A61M 2205/3584; A61M 2205/36

(57) ABSTRACT

The present disclosure relates to a portable vaporizer capable of vaporizing liquid medicament such as herbal oils or therapeutic liquids into a vapor for pulmonary administration to treat a variety of medical conditions, the vaporizer can include a housing having a proximal end and a distal end, wherein the housing including an opening at the proximal end defining a mouth piece, a liquid reservoir disposed in the housing to hold a liquid medicament to be vaporized, and a vaporization unit. The portable vaporizer can dispense metered quantity of vaporized liquid material at the mouthpiece by operation of the vaporization unit, and it can be readily used in the event of a sudden onset of serious medical symptoms such as headaches, chronic pain or anxiety. The portable vaporizer can further include a communication module to communicate with external functional units or networks for recording the dosage history or other functions.

### TECHNICAL FIELD

The present disclosure relates generally to the technical field of therapeutic vaporizers. In particular, the present disclosure pertains to a portable vaporizer that vaporizes medicated liquids or herbal oils for inhalation to treat a variety of medical conditions.

### BACKGROUND

Background description includes information that may be useful in understanding the present invention. It is not an admission that any of the information provided herein is prior art or relevant to the presently claimed invention, or that any publication specifically or implicitly referenced is prior art.

Vaporizers are used to vaporize medicated liquids or herbal materials for the purpose of inhalation. Vaporizers are often used as a therapeutic device to provide vaporized medications, as an alternative to oral and intravenous administration of medications. A variety of vaporizers and inhalation devices for vaporizing herbal oils and therapeutic liquids for pulmonary delivery have been developed and some of them are commercially available. Although effective in vaporizing vaporizable substances, many of the prior art vaporizing devices suffer from a number of disadvantages which include not being discrete, being limited in use, being limited in dose control, being bulky, being inconvenient to use, and emitting offensive smell.

An example of a vaporizer is a nebulizer or an atomizer. Such a device turns solid or liquid substance into aerosol using air, oxygen or a propellant gas. The aerosol can then be inhaled and instantly delivered to the lungs. The most common nebulizer is an “asthma inhaler” or “allergy inhaler” that delivers medication directly to the bronchi to alleviate the constriction caused by an allergic reaction or an asthma attack. Because they do not require heating units, nebulizers are often small, portable hand-held devices. However, nebulizers cannot be manually refilled. As a result, they are either single-use or refillable using stationary pumps. Most often, nebulizers come pre-filled with a particular medication and are discarded after use.

Another disadvantage of the prior art devices is that they provide no provision for communication with external networks or other functional units for recording critical data relating to usage of vaporizer device and dosage of medicinal product for subsequent clinical analysis.

Accordingly, there exists a need in the art for a vaporizer that is very portable, discreet, provides metered dose of vaporized medications, economical to build, convenient to operation and eliminates production and release of unwanted smoke fumes. There also exists a need for a vaporizer that enables communication with external networks or other functional units for recording usage of vaporizer device and/or the dosage history. The present invention satisfies the existing needs, as well as others, and generally overcomes the deficiencies found in the prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

### Objects of the Invention

An object of the present disclosure is to provide a therapeutic vaporizer that is portable.

Another object of the present disclosure is to provide a vaporizer that is discrete.

Another object of the present disclosure is to provide a vaporizer that provides metered dose of vaporized medications.

Another object of the present disclosure is to provide a vaporizer that is economical to build.

Another object of the present disclosure is to provide a vaporizer that is convenient to operate.

Another object of the present disclosure is to provide a vaporizer that eliminates production and release of unwanted smoke fumes.

### SUMMARY

The present disclosure relates generally to the technical field of therapeutic vaporizers. In particular, the present disclosure pertains to a portable vaporizer that vaporizes medicated liquids or herbal oils for inhalation to treat a variety of medical conditions.

An aspect of the present disclosure provides a portable vaporizer capable of vaporizing a medicament for pulmonary administration, the portable vaporizer including: a housing having a proximal end and a distal end, wherein the housing comprises an opening at the proximal end defining a mouthpiece; a reservoir disposed in the housing to hold the medicament to be vaporized; a vaporization unit including a dispensing unit configured to dispense a metered quantity of the medicament from the reservoir; and a heating unit configured to vaporize the medicament to produce a metered quantity of vaporized medicament at the mouthpiece.

In an embodiment, the housing includes a power module capable of supplying electric power to the heating unit sufficient to vaporize the medicament. In an embodiment, a push button is disposed on the housing and is in electrical communication with the power module for activation of the vaporizer. In an embodiment, the portable vaporizer is configured to communicate with any or a combination of an external network and a functional unit for any or a combination of recording of clinical data and transmission of the clinical data. In an embodiment, the clinical data is selected from a group including dosage of the medicament administered, dosage administration time, dosage administration date, number of doses administered, usage of the vaporizer and combination thereof. In an embodiment, the any or a combination of the external network and the functional unit is configured to receive any or a combination of a patient feedback and a patient data from external device(s). In an embodiment, the any or a combination of the external network and the functional unit is configured to effect any or a combination of (a) calculation of quantity of the medicament to be vaporized based on any or a combination of the clinical data, the patient feedback and the patient data and (b) transmission of information pertaining to quantity of the medicament to be vaporized to the portable vaporizer. In an embodiment, the portable vaporizer is configured to vaporize the medicament for pulmonary administration based on the information pertaining to the quantity of the medicament to be vaporized.

In an embodiment, the portable vaporizer is configured with a communication port to effect connection of the vaporizer with any or a combination of the external network and the functional unit. In an embodiment, the portable vaporizer is configured to connect wirelessly with any or a combination of the external network and the functional unit. In an embodiment, the portable vaporizer further includes at least one indicator on the housing to indicate functionality of the vaporizer. In an embodiment, the vaporizer can vaporize a variety of herbal oils and dispense metered doses of vaporized herbal oil for inhalation to treat a variety of medical conditions.

According to embodiments of the present disclosure, the portable vaporizer can be in communication with external networks or other functional units for recording clinical data including dosage time, date and number of doses administered, and the usage of the vaporizer apparatus for subsequent analyses. Thus, the portable vaporizer and the external functional systems may communicate and provide information and/or instructions one to the other.

In an embodiment, the vaporizer can be configured to include a communication port that can facilitate connection of the vaporizer to external networks or other functional units through a communication cable. In another embodiment, the vaporizer can be configured such that it can be wirelessly connected to external functional systems/units over a network. In an embodiment, any suitable wireless communication protocol can be used, including but not limited to, Wi-Fi, Bluetooth, GSM and the like.

According to embodiments, the portable vaporizer can include a plurality of indicator lights on the housing to illuminate a predetermined color to indicate the functionality of the vaporizer.

The portable vaporizer disclosed herein can fit within a user's pocket, purse or handbag, and it can be readily used in the event of a sudden onset of serious medical symptoms such as headaches, chronic pain or anxiety.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIG. 1 is a schematic block diagram of a portable vaporizer representing an embodiment of the present disclosure.

FIG. 2 is a perspective view of a portable vaporizer in accordance with embodiments of the present disclosure.

FIG. 3 is a perspective view of an exemplary portable vaporizer for vaporizing a metered quantity of liquid medicament in accordance with embodiments of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Various terms as used herein are shown below. To the extent a term used in a claim is not defined below, it should be given the broadest definition persons in the pertinent art have given that term as reflected in printed publications and issued patents at the time of filing.

The present disclosure relates generally to the technical field of therapeutic vaporizers. In particular, the present disclosure pertains to a portable vaporizer that vaporizes medicated liquids or herbal oils for inhalation to treat a variety of medical conditions.

An aspect of the present disclosure provides a portable vaporizer capable of vaporizing a medicament for pulmonary administration, the portable vaporizer comprising: a housing having a proximal end and a distal end, wherein the housing comprises an opening at the proximal end defining a mouthpiece; a reservoir disposed in the housing to hold the medicament to be vaporized; a vaporization unit comprising a dispensing unit configured to dispense a metered quantity of the medicament from the reservoir; and a heating unit configured to vaporize the medicament to produce a metered quantity of vaporized medicament at the mouthpiece.

In an embodiment, the housing comprises a power module capable of supplying electric power to the heating unit sufficient to vaporize the medicament. In an embodiment, a push button is disposed on the housing and is in electrical communication with the power module for activation of the vaporizer. In an embodiment, the portable vaporizer is configured to communicate with any or a combination of an external network and a functional unit for any or a combination of recording of clinical data and transmission of the clinical data. In an embodiment, the clinical data is selected from a group comprising dosage of the medicament administered, dosage administration time, dosage administration date, number of doses administered, usage of the vaporizer and combination thereof.

In an embodiment, the any or a combination of the external network and the functional unit is configured to receive any or a combination of a patient feedback and a patient data from external device(s). In an exemplary embodiment, the patient feedback is related to level of pain associated with his/her therapeutic condition on days when he/she exercises. In another exemplary embodiment, the patient data from external device(s) can be related to barometric pressure of the area where the patient is located or any other geographic data. However, a person skilled in the art would appreciate that the patient feedback or the patient data received from any external device(s) may include any other data that may find utility in arriving at an appropriate medication dosing or which may find utility in any clinical setup without departing from the scope and spirit of the present invention.

In an embodiment, the any or a combination of the external network and the functional unit is configured to effect any or a combination of (a) calculation of quantity of the medicament to be vaporized based on any or a combination of the clinical data, the patient feedback and the patient data and (b) transmission of information pertaining to quantity of the medicament to be vaporized to the portable vaporizer. In an embodiment, the external network is configured to calculate medication doses for a patient based on any or a combination of the patient feedback, the patient data and the clinical data, for maximum efficacy. In an embodiment, the portable vaporizer is configured to vaporize the medicament for pulmonary administration based on the information pertaining to the quantity of the medicament to be vaporized.

In an embodiment, the portable vaporizer is configured with a communication port to effect connection of the vaporizer with any or a combination of the external network and the functional unit. In an embodiment, the portable vaporizer is configured to connect wirelessly with any or a combination of the external network and the functional unit. In an embodiment, the portable vaporizer further comprises at least one indicator on the housing to indicate functionality of the vaporizer. In an embodiment, the vaporizer can vaporize a variety of herbal oils and dispense metered doses of vaporized herbal oil for inhalation to treat a variety of medical conditions. In an embodiment, the vaporizer disclosed herein can vaporize a variety of herbal oils and dispense metered doses of vaporized herbal oil for inhalation to treat a variety of medical conditions.

According to embodiments of the present disclosure, the portable vaporizer can be in communication with external networks or other functional units for recording clinical data including dosage time, date and number of doses administered, and the usage of the vaporizer apparatus for subsequent analyses. Thus, the portable vaporizer and the external functional systems may communicate and provide information and/or instructions one to the other.

In an embodiment, the vaporizer can be configured to include a communication port that can facilitate connection of the vaporizer to external networks or other functional units through a communication cable. In another embodiment, the vaporizer can be configured such that it can be wirelessly connected to external functional systems/units over a network. In an embodiment, any suitable wireless communication protocol can be used, including but not limited to, Wi-Fi, Bluetooth, GSM and the like.

According to embodiments, the portable vaporizer can include a plurality of indicators on the housing to illuminate a predetermined color to indicate the functionality of the vaporizer. The portable vaporizer disclosed herein can fit within a user's pocket, purse or handbag, and it can be readily used in the event of a sudden onset of serious medical symptoms such as headaches, chronic pain or anxiety. In an embodiment, body of the portable vaporizer can be in any suitable shape. In a preferred embodiment, body of the portable vaporizer is in the shape of a standard-sized cellular phone. As used herein the term “liquid medicament” encompasses all types of vaporizable liquid substances that have a beneficial or therapeutic effect. Non-limiting examples of liquid medicament suitable for use in the present invention can include any of the oils derived from medicinal herbs and biologically active agents in liquid form.

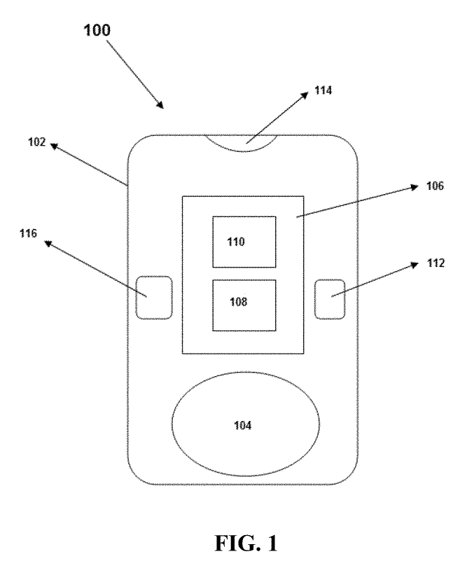

Referring to FIG. 1, there is shown a schematic block diagram of a portable vaporizer 100 representing an embodiment of the present disclosure. The vaporizer 100 can include a housing 102, a liquid reservoir 104, a vaporization unit 106, a power module 112, a mouth piece 114 and a communication module 116. The housing 102 can be formed by a plurality of housing walls and it can provide a structure for the vaporizer apparatus. The housing 102 may be composed of plastic, metal, fiberboard, ceramic or a combination of these materials. The liquid reservoir 104 can be configured to hold a volume of a liquid medicament that is to be vaporized. In an embodiment, the reservoir is configured to hold a volume of liquid medicament sufficient to provide a maximum of ten doses. The vaporization unit 106 can include a liquid dispensing unit 108 and a heating unit 110. The liquid dispensing unit 106 can dispense a metered quantity of liquid medicament from the reservoir 104 to the heating unit 110 for vaporization. Preferably, the dispensing unit 106 can be configured to dispense a single drop of a fixed volume upon each actuation of the vaporizer apparatus. The heating unit 110 can be constructed from a variety of heating coils with different configurations and capacities. In an embodiment, the heating coils can be made up of a variety of metallic alloys, nickel-chrome, other types of metals, or metal based composites that have a general low thermal resistivity and are generally safe to pass air through for human consumption. In further embodiments, the heating unit 110 may contain heating elements that may be in the form of a plate or other shape, and may be located within a piece of glass or in close proximity to the mouthpiece 114, but yet still able to effectively transfer heat. The heating unit 110 can heat a liquid to produce vapors, which in turn can be directed toward the mouthpiece 114 where a user can inhale the vaporized medicament. Hence, each time the vaporizer 100 is actuated, a metered dose of vaporized medicament can be dispensed at the mouthpiece 114 by operation of the vaporization unit 106. In an embodiment, the mouthpiece 114 is non-conductive so that a user can safely place his/her mouth on the mouthpiece to create a “suction effect” and inhale the generated vapors. In an embodiment, the mouthpiece 114 is made with glass. In another embodiment, the mouthpiece 114 is made from a non-conductive polymer or any other non-conductive material that resists the transfer of heat through the material. In an embodiment, the mouthpiece 114 can be adjusted to any length.

The housing 102 can further include a power module 112 capable of supplying electric power to the heating element 110 sufficient to vaporize a liquid medicament. The power module 112 can preferably be a battery, rechargeable battery and the like. In an embodiment, the battery can be lithium-ion based battery or silica based battery or any other battery, as known to a person skilled in the art. In an embodiment, an adjustable voltage regulator can be provided to prevent excess battery drain. This is an advantageous feature of the instant invention over vaporizers known in the art that utilize fuel-based systems to heat the air to the vaporizing temperature because such fuel-based systems produce foul-tasting by-products and are generally loud and bring attention to the device. In an embodiment, the portable vaporizer 100 may include an optional charging port. The charging port may be located anywhere on the body/housing of the portable vaporizer. A push button can be disposed on the housing 102 and it can be in electrical communication with the power module 112 for activation of the vaporizer 100. A push button may be configured such that it turns the power on when pressed and off when pressed again.

In an embodiment, the heating element 110 can heat a metered quantity of a liquid medicament dispensed from the dispensing unit 108 to a temperature ranging from 160 to 220° C. to produce vapors. In other embodiments, the heating element 110 may be supplied power to reach a predetermined temperature or may have a display indicating to the user what the temperature is or that the device is ready for use.

The vaporizer 100 can further include a communication module 116 that can be configured to connect the vaporizer with external network(s) and/or functional units in various modes for recording clinical data including dosage time, date and number of doses administered, the usage of the vaporizer and the likes for subsequent analysis. Non-limiting examples of functional units can include computing devices, mobile devices, networks, databases and other types of applications/software. Communication between the portable vaporizer 100 and the external functional system may be accomplished through any suitable communication channels, such as, but not limited to, a telephone network, internet, online communications, off-line communications, wireless communications, networked devices and the like. Thus, the portable vaporizer 100 and the external network(s)/functional unit(s) may communicate and provide information and/or instructions one to the other. In an embodiment, any suitable wireless communication protocol can be used, including but not limited to, Wi-Fi, Bluetooth, GSM and the like.

In an embodiment, the vaporizer disclosed herein can be provided with a communication port that can facilitate connection of the vaporizer to the external network(s)/functional unit(s) through a communication cable. In an alternative embodiment, the vaporizer can be wirelessly connected to the external network(s)/functional unit(s) over a network. In an embodiment, any suitable wireless communication protocol can be used, including but not limited to, Wi-Fi, Bluetooth, GSM and the like.

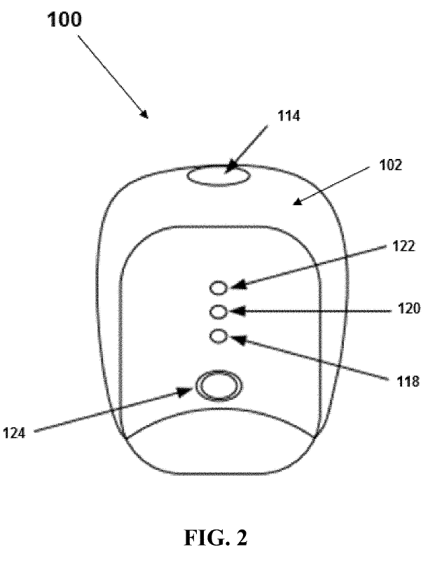

According to an embodiment, the vaporizer disclosed herein can include one or a plurality of lights to indicate the functionality of the vaporizer. FIG. 2 shows perspective view of a portable vaporizer 200 designed to provide metered doses of vaporized liquid medicament for inhalation. The vaporizer 200 includes three indicator lights on the housing 102 for providing an indication of the operation of vaporizer 200. The indicator light 118 may illuminate to indicate if the vaporizer 100 is powered on. The indicator light 120 may illuminate when the unit is ready to vaporize a liquid or is actively vaporizing a liquid medicament. The indicator light 122 may illuminate when a predetermined dosage limit is exceeded by a user, and to provide an alert to the user to connect the vaporizer to an external functional unit or network to view the dosage information. Any other structure/construction/configuration of the lights or the purpose(s) for which they are used is completely within the scope of the present disclosure.

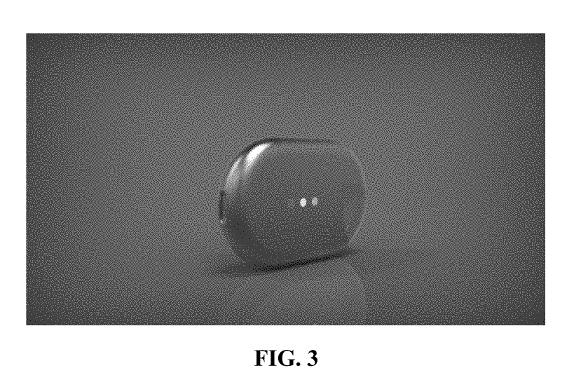

The configuration of the vaporizer 100 ensures that a vaporized liquid medicament exits the vaporizer through the mouthpiece 114 regardless of the orientation of the vaporizer, thereby enabling a user to operate the vaporizer in a wide variety of orientations. The vaporizer 100 can be constructed such that it can be handheld and operated with convenience to the user. The portable vaporizer disclosed herein can fit within a user's pocket, purse or handbag, and it can be used in the event of a sudden onset of a serious medical symptom such as headaches, chronic pain or anxiety. A fully constructed exemplary portable vaporizer for producing metered doses of vaporized medicament is shown in FIG. 3.

In an embodiment, the portable vaporizer of the present disclosure can be utilized by any individual suffering from COPD, asthma, bronchitis, or any other pulmonary condition known to a person skilled in the art. In an embodiment, the portable vaporizer of the present disclosure can also be configured to function as an essential oil vaporizer.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### Advantages of the Invention

The present disclosure provides for a therapeutic vaporizer that is portable.

The present disclosure provides for a vaporizer that is discrete.

The present disclosure provides for a vaporizer that provides metered dose of vaporized medications.

The present disclosure provides for a vaporizer that is economical to build.

The present disclosure provides for a vaporizer that is convenient to operate.

The present disclosure provides for a vaporizer that eliminates production and release of unwanted smoke fumes.

We claim:

1. A portable vaporizer capable of vaporizing a medicament for pulmonary administration, the portable vaporizer comprising:
a housing having a proximal end and a distal end, wherein the housing comprises an opening at the proximal end defining a mouthpiece;
a reservoir disposed in the housing to hold the medicament to be vaporized;
a vaporization unit comprising a dispensing unit configured to dispense a metered quantity of the medicament from the reservoir; and
a heating unit configured to vaporize the medicament to produce a metered quantity of vaporized medicament at the mouthpiece.

2. The portable vaporizer as claimed in claim 1, wherein the housing comprises a power module capable of supplying electric power to the heating unit sufficient to vaporize the medicament.

3. The portable vaporizer as claimed in claim 1, wherein a push button is disposed on the housing and is in electrical communication with the power module for activation of the vaporizer.

4. The portable vaporizer as claimed in claim 1, wherein the portable vaporizer is configured to communicate with any or a combination of an external network and a functional unit for any or a combination of recording of clinical data and transmission of the clinical data.

5. The portable vaporizer as claimed in claim 4, wherein the clinical data is selected from a group comprising dosage of the medicament administered, dosage administration time, dosage administration date, number of doses administered, usage of the vaporizer and combination thereof.

6. The portable vaporizer as claimed in claim 4, wherein the portable vaporizer is configured with a communication port to effect connection of the vaporizer with any or a combination of the external network and the functional unit.

7. The portable vaporizer as claimed in claim 4, wherein the portable vaporizer is configured to connect wirelessly with any or a combination of the external network and the functional unit.

8. The portable vaporizer as claimed in claim 1, wherein the portable vaporizer further comprises at least one indicator on the housing to indicate functionality of the vaporizer.

9. The portable vaporizer as claimed in claim 1, wherein the any or a combination of the external network and the functional unit is configured to receive any or a combination of a patient feedback and a patient data from one or a plurality of external device.

10. The portable vaporizer as claimed in claim 1, wherein the any or a combination of the external network and the functional unit is further configured to effect any or a combination of (a) calculation of quantity of the medicament to be vaporized based on any or a combination of the clinical data, the patient feedback and the patient data and (b) transmission of information pertaining to quantity of the medicament to be vaporized to the portable vaporizer.

11. The portable vaporizer as claimed in claim 10, wherein the portable vaporizer is configured to vaporize the medicament for pulmonary administration based on the transmitted information pertaining to the quantity of the medicament to be vaporized.
